(19) United States
(12) Patent Application Publication
(10) Pub. No.: US 2007/0156207 A1
(43) Pub. Date: Jul. 5, 2007

(54) Expanding single channel stimulator capability on multi-area stimulation programs

(76) Inventors: Sridhar Kothandaraman, Valencia, CA (US); Kerry Bradley, Glendale, CA (US)

Correspondence Address: Vista IP Law Group LLP, 2040 MAIN STREET, 9TH FLOOR, IRVINE, CA, 92614, US

(21) Appl. No.: 11/324,811

(22) Filed: Jan. 4, 2006

(51) Int. Cl. A61N 1/36

(52) U.S. Cl. 607/66; 607/46; 607/117

(57) ABSTRACT

Tissue stimulation systems generally include a pulse generating device for generating electrical stimulation pulses, at least one implanted lead including at least one electrode for delivering the electrical stimulation pulses generated by the pulse generating device, and a programmer capable of communicating with the pulse generating device. In tissue stimulation systems, two or more electrical stimulation combinations may be delivered to a patient simultaneously through a reduced number of channels. Systems and methods described herein may combine the two or more stimulation combinations into a reduced number of new stimulation combinations for delivery of the stimulation combinations over a reduced number of channels.

### BACKGROUND OF THE INVENTION

The present invention relates to tissue stimulation systems and more particularly to applying electrical stimulation pulses to a patient through a single channel to treat separate areas of the patient's body.

One example of a stimulation system is a spinal cord stimulation (“SCS”) system. Spinal cord stimulation is a well accepted clinical method for reducing pain in certain populations of patients. An SCS system typically includes an Implantable Pulse Generator (IPG) or a radio-frequency (RF) transmitter and receiver, electrodes, electrode leads, and when necessary, lead extensions. The electrodes are implanted along the dura of the spinal cord, and the IPG or RF transmitter generates electrical pulses that are delivered, through the electrodes, to the dorsal column and dorsal root fibers within the spinal cord. Individual electrodes are arranged in a desired pattern and spacing in order to create an electrode array. Individual wires within one or more electrode leads connect with each electrode in the array. The electrode leads exit the spinal column and attach to one or more electrode lead extensions, when necessary. The electrode leads or extensions are typically tunneled around the torso of the patient to a subcutaneous pocket where the IPG or RF-receiver is implanted.

Spinal cord stimulators and other stimulation systems are known in the art. For example, an implantable electronic stimulator is disclosed in U.S. Pat. No. 3,646,940 issued Mar. 7, 1972 for “Implantable Electronic Stimulator Electrode and Method” that provides timed sequenced electrical impulses to a plurality of electrodes. As another example, U.S. Pat. No. 3,724,467 issued Apr. 3, 1973 for “Electrode Implant for the Neuro-Stimulation of the Spinal Cord,” teaches an electrode implant for the neuro-stimulation of the spinal cord. A relatively thin and flexible strip of physiologically inert plastic is provided as a carrier on which a plurality of electrodes are formed. The electrodes are connected by leads to an RF receiver, which is also implanted.

Exemplary IPGs suitable for use include, but are not limited to, those disclosed in U.S. Pat. Nos. 6,381,496, 6,553,263, and 6,760,626. Exemplary spinal cord stimulators suitable for use include, but are not limited to, those disclosed in U.S. Pat. Nos. 5,501,703, 6,487,446, and 6,516,227.

In U.S. Pat. No. 3,822,708, issued Jul. 9, 1974 for “Electrical Spinal Cord Stimulating Device and Method for Management of Pain,” another type of electrical spinal cord stimulation device is taught. The device disclosed in the '708 patent has five aligned electrodes, which are positioned longitudinally on the spinal cord. Electrical pulses applied to the electrodes block perceived intractable pain, while allowing passage of other sensations. A patient operated switch allows the patient to adjust the stimulation parameters.

An SCS system treats chronic pain by providing electrical stimulation pulses through the electrodes of an electrode array located at the distal end of a lead placed epidurally adjacent to a patient's spinal cord. An electrode combination represents the polarity (positive, negative, or zero) of each electrode, and for certain SCS systems with such capabilities, may also refer to the relative percentage of the current or voltage provided through each of the electrodes. Electrode arrays used with known SCS systems may employ between 1 and 16 electrodes on a lead. Additionally, the case or can of the pulse generator or RF receiver may act as an electrode. Electrodes are selectively programmed to act as anodes, cathodes, or left off. Electrodes that are programmed to act as anodes or cathodes are referred to as “active” electrodes, whereas electrodes programmed off are referred to as “inactive” electrodes. The number of electrodes available, combined with the ability to generate a variety of complex stimulation pulses, presents a huge selection of electrode combinations and stimulation parameters to the user.

Other parameters that may be controlled or varied in SCS include the frequency of pulses provided through the electrode array, pulse width, and the amplitude of pulses delivered. Amplitude may be measured in milliamps, volts, etc., as appropriate, depending on whether the system provides stimulation from current sources or voltage sources. A stimulation combination refers to which electrodes are active and the stimulation parameters defining the stimulation delivered by the active electrodes.

Programming processes are described in U.S. Pat. No. 6,622,048, herein incorporated by reference in its entirety. A stimulation programmer is utilized to instruct the pulse generating device to generate electrical stimulation pulses in accordance with selected parameters or stimulation sets. A stimulation programmer may be used to program the stimulator by a technician or clinician attending the patient, or, in some cases, by the patient his/herself. A stimulation programmer may be used in several scenarios. For example, when an SCS system is implanted, a procedure is performed to assure that the leads and/or electrodes are properly implanted in effective locations in the body. A navigation session is a procedure to select one or more effective stimulation sets for a particular patient. Such a session occurs after the leads and/or electrodes are implanted into a patient.

In SCS, the various electrodes on the implanted lead may stimulate different areas in the spinal column. This is due to the relative orientations of the electrodes to the nerves in a spinal column and the distribution of the current or voltage applied to the electrodes resulting in a combination of cathodes, anodes, and inactive electrodes. For example, electrodes 1, 3, and 5 of an eight (8) electrode lead may be programmed as an anode, a cathode, and an anode respectively (i.e., +, 0, −, 0, +, 0, 0, 0). Depending on the position of the implanted lead, this stimulation combination may supply stimulation that induces paresthesia in the upper leg area. Stimulation of different nerves in the spinal column results in different therapeutic effects on the patient's body. One such therapeutic effect is a paresthesia sensation on the patient's body. An “area” refers to a region or regions of the body in which paresthesia is felt due to a stimulation combination.

Some SCS systems employ multiple channels, wherein each channel uses a particular stimulation combination to provide paresthesia to a specific area. The use of a multi-channel stimulator allows for treatment of multiple, distinct areas of the body using a single pulse generator or RF receiver, resulting in paresthesia more comfortably mapped to the targeted region(s) of pain. For example, if a patient has pain in only the lower leg during the day, but in both the upper and lower leg while sleeping, a suitable stimulation combination can be found to treat the pain in the lower leg and stored as channel 1. Then, a suitable stimulation combination can be found to treat the pain in both the upper and lower leg while reclined, and this combination can be stored as channel 2. Once programmed, the patient is able to select which channel to activate at any particular time. Multiple channels may also be used at one time, as described in more detail below.

A channel generally refers to a timing generator, wherein the stimulator generates a pulse with individually controllable pulse amplitude, pulse width, and pulse rate to the active electrodes that correspond with paresthesia for a particular “area”. In order to cover multiple areas with multiple channels, while maintaining the sensation of simultaneous paresthesia, the timing generators may use interleaved pulsing techniques, where the stimulating pulses are separated in time. The use of interleaved pulses prevents temporal summation in the nerves, but are generated close enough in time such that there is no sensation of rapidly shifting paresthesias perceived by the patient, instead giving the patient the perception that both channels are “on” simultaneously. Less sophisticated systems may attempt to replicate the effects of multiple channels by cycling through various stimulation combinations on a single channel. The present invention may also benefit systems that use this method to imitate true “channels”.

The use of multiple channels may be advantageous due to the flexibility of programming each area individually. However, applying stimulation pulses to a patient using multiple channels may be an inefficient use of stimulator power supply. Since more net pulses per second may be delivered by a stimulator using multiple channels, there can be an increased drain on the battery.

Thus, it is advantageous to expand programming capabilities to facilitate reducing the number of channels (when possible), while still providing stimulation to multiple areas of the body. It is also advantageous to facilitate organizing stimulation pulses into as few channels as possible to increase the efficiency of stimulation therapies.

### SUMMARY OF THE INVENTION

The present invention addresses the above and other advantages by providing a method of facilitating combining multiple stimulation channels into a reduced number of channels, preferably a single channel.

An embodiment includes a method of reducing the number of stimulation channels used for stimulation therapy. The method may include: (1) determining two or more stimulation combinations, wherein each stimulation combination is capable of inducing a corresponding therapeutic effect in a patient's body and wherein each stimulation combination is deliverable to the patient via a separate channel; (2) evaluating the two or more stimulation combinations to determine if the two or more combinations may be combined into a reduced number of new stimulation combinations; (3) combining the two or more stimulation combinations into a reduced number of new stimulation combinations; and (4) storing the new stimulation combinations for application to the patient via a reduced number of stimulation channels.

In the evaluating step, if the one or more stimulation combinations share an electrode, an algorithm may determine which stimulation combination uses the shared electrode for a greater percentage of current distribution and may assign the shared electrode that percentage of current distribution. The evaluating step may also include evaluating the effect of the combined stimulation fields generated by each of the one or more stimulation combinations and measuring impedance and/or threshold values.

The combining step may include a redistributing of current over the combined active electrodes, such as for instance, in a pro rata manner. Further steps of the method include applying stimulation to the patient according to the reduced number of channels.

The new stimulation combinations may correspond to two or more corresponding therapeutic effects on the patient's body. Such therapeutic effects may be the sensation of paresthesia in two or more areas of the patient's body, which areas may or may not be overlapping.

Another method of reducing the number of stimulation channels for a stimulation therapy may include: (1) determining two or more stimulation combinations, wherein each stimulation combination is capable of inducing a corresponding therapeutic effect in a patient's body and wherein each stimulation combination is deliverable to the patient via a separate channel; (2) evaluating the two or more stimulation combinations to determine if the two or more combinations may be combined into a reduced number of new stimulation combinations; (3) combining the two or more stimulation combinations into a reduced number of new stimulation combinations; (4) applying stimulation to the patient according to the new stimulation combinations via a reduced number of stimulation channels; and (5) evaluating the new stimulation combinations.

A tissue stimulation system may include: (1) a pulse generating device for generating electrical stimulation pulses; (2) at least one implanted lead including at least one electrode for delivering the electrical stimulation pulses generated by the pulse generating device; and (3) a programmer for programming two or more stimulation combinations to be generated by the pulse generating device to be delivered via one or more stimulation channels, wherein said programmer is capable of combining the stimulation combinations into a reduced number of new stimulation combinations to be delivered by the at least one electrode over a reduced number of stimulation channels.

The at least one implanted lead of the system may be implanted near the patient's spinal column, such as in an SCS system. The programmer may be a separate device or may be incorporated into the pulse generating device. The pulse generating device may be an IPG. The programmer is capable of combing the stimulation combinations into a reduced number of new stimulation combinations during or after the programming of the individual stimulation combinations.

### BRIEF DESCRIPTION OF THE DRAWINGS

The above and other aspects of the present invention will be more apparent from the following more particular description thereof, presented in conjunction with the following drawings wherein:

FIG. 1 depicts a Spinal Cord Stimulation (SCS) system, as an example of a tissue stimulation system.

FIG. 2 depicts the SCS system of FIG. 1 implanted in a spinal column.

FIG. 3 is a timing diagram that depicts representative waveforms that may be applied to various ones of electrodes of the electrode arrays through one or more stimulus channels.

FIG. 4 depicts a process of determining a reduced number (e.g., a single) channel for use in a stimulation therapy.

FIG. 5 is a timing diagram that depicts representative waveforms wherein two stimulation combinations from FIG. 3 have been combined into one channel.

### DETAILED DESCRIPTION OF THE INVENTION

It is to be understood that this invention is not limited to the particular devices, compositions, methodologies or protocols described, as these may vary. It is also to be understood that the terminology used in the description is for the purpose of describing the particular versions or embodiments only, and is not intended to limit the scope of the present invention which will be limited only by the appended claims.

It must also be noted that as used herein and in the appended claims, the singular forms “a”, “an”, and “the” include plural reference unless the context clearly dictates otherwise. Thus, for example, reference to an “electrode” is a reference to one or more electrodes and equivalents thereof known to those skilled in the art, and so forth. Unless defined otherwise, all technical and scientific terms used herein have the same meanings as commonly understood by one of ordinary skill in the art. Although any methods, devices, and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

A Spinal Cord Stimulation (SCS) system will be used herein as an example of such a tissue stimulation system. The various components of an exemplary SCS system may include an implantable pulse generator (IPG) and programmer used with such system. Implantable components may include an implantable pulse generator, one or more electrode arrays/leads, and (as needed) one or more extensions to connect the array(s)/lead(s) to the IPG. Such implantable components, external devices and circuitry are more fully described in U.S. Pat. No. 6,622,048. Alternatively, a system comprised of an implanted RF receiver and external transmitter, as a pulse generating device in place of an IPG, may be used. The tissue stimulator may include a rechargeable or replenishable energy source, such as a rechargeable battery.

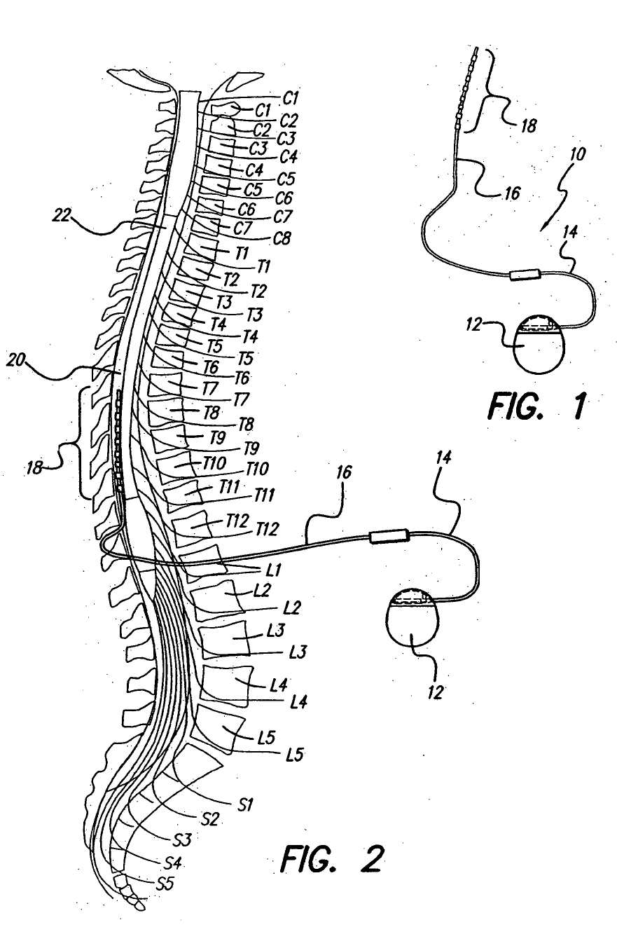

An exemplary Spinal Cord Stimulation (SCS) system 10 is shown in FIG. 1. SCS system 10 comprises an Implantable Pulse Generator (IPG) 12, an optional lead extension 14, an electrode lead 16, and an electrode array 18. The IPG 12 generates stimulation current for implanted electrodes that make up the electrode array 18. When needed, a proximal end of the lead extension 14 is removably connected to the IPG 12 and a distal end of the lead extension 14 is removably connected to a proximal end of the electrode lead 16. Alternatively, a proximal end of lead 16 is attached directly to the IPG 12. Electrode array 18 is formed on a distal end of the electrode lead 16. The in-series combination of the optional lead extension 14 and the electrode lead 16, carry the stimulation current from the IPG 12 to the electrode array 18.

The SCS system 10 described in FIG. 1 above is depicted implanted in the epidural space 20 in FIG. 2. The electrode array 18 is implanted at the site of nerve fibers that are the target of stimulation, e.g., along the spinal cord. Due to the lack of space near the location where the electrode lead 16 exits the spinal column, the IPG 12 is generally implanted in the abdomen or above the buttocks. When needed, the lead extension 14 facilitates locating the IPG 12 away from the electrode lead exit point. Other examples of SCS systems that may be used with the present invention are described in U.S. Pat. Nos. 6,516,227, and 6,393,325 and related applications and issued patents. It is to be emphasized, however, that the invention herein described may be used with many different types of stimulation systems, and is not limited to use with the representative SCS system.

An implantable tissue stimulator generally receives an RF or other control signal from an external source, e.g., from a programmer. The programmer may be incorporated into the IPG or other pulse generating device or it may be a separate device. Thus, the programmer may be implanted in or external to the patient. Additionally, systems may apportion control of the pulse generating device over several devices. For example, control may be shared between the pulse generating device itself and a programmer. Such variations in hardware are known in the art.

Typically, the programmer sends an operating program to the tissue stimulator, generally causing an electrical stimulation current to be applied to one or more electrodes, E1, E2, E3, . . . En, associated with the stimulator. The operating program consists of one or more sets of stimulation combinations, and each stimulation combination specifies which of the electrodes within the multiplicity of electrodes E1, E2, E3, . . . En included within an array of electrodes, are turned ON as an anode or cathode, or turned OFF. If an electrode is turned ON, the operating program also includes characterization data for each electrode, such as the amplitude, pulse width, and frequency of stimulation pulses delivered by that stimulation combination. This characterization data may be preprogrammed into the processor, or it may be set through use of manual selection input/output (I/O) devices, which devices may be implemented in hardware (e.g., slide switches) or software (e.g., simulated slide switches that appear on the display screen of the programmer).

A given stimulation combination may be delivered continuously or for a specified amount of time. Additionally, several different stimulation combinations may be delivered at the same time on different channels, wherein the tissue stimulator staggers the delivery of pulses from each channel and ensures that pulses from different channels are not delivered at exactly the same time.

In some programming modes, an indifferent or return electrode, Eg, which may in fact form part of the case or housing of the implantable pulse generator, may be used with individual ones of the electrodes E1, E2, E3, . . . En so as to provide “monopolar” stimulation. Stimulation currents must always be applied through two or more electrodes, with at least one electrode functioning as an anode and with at least one electrode functioning as a cathode, so that the stimulation current may flow into the tissue to be stimulated through one path and return therefrom through another path.

The following issued United States patents, each of which is incorporated herein by reference, provide additional detail associated with implantable tissue stimulators, programming such stimulators, and the use of stimulation pulses in a bipolar, monopolar or other stimulation mode: U.S. Pat. Nos. 5,776,172; 5,649,970; 5,626,629; and 5,601,617. A stimulation programmer may interface with a user device and also with the implanted pulse generator. Programmers may be in the form of a conventional PC, a laptop, a tablet, a PDA, a monitor, a hand-held device, and any other suitable computing means.

Two or more separately programmable channels are available in some SCS systems. A “channel” is defined as a group of electrodes that receive a specified pattern or sequence of stimulus pulses. Thus, where more than one “channel” is available, each channel may be programmed to provide its own specified pattern or sequence of stimulus pulses to its defined electrodes. In operation, all of the stimulus patterns applied through all of the channels of such multi-channel system thus combine to provide an overall stimulation pattern that is applied to the tissue exposed to the individual electrodes of the SCS system.

There are many instances when it is advantageous to have multiple channels, such as for example, the tissue stimulator described in U.S. Pat. No. 6,516,227, herein incorporated by reference in its entirety. For example, left and right sides or upper and lower extremities may require stimulation to be applied by anodes and cathodes in different locations along an electrode array, and may require different stimulus parameter settings. Thus, one extremity may require short powerful stimulation pulses through a few electrodes located on one end of the array, while another part of the body may require a more moderate pulse distributed among multiple electrodes located throughout the array. Low back pain typically requires a special stimulation site and stimulation parameters. Therefore, having multiple channels that may be connected to multiple electrodes, positioned within one or more electrode arrays, so as to cover more tissue/nerve area, greatly facilitates providing the type of stimulation patterns and stimulation parameters needed to treat a particular patient.

There are SCS systems, however, that only provide a single channel. Additionally, operation of several distinct channels may result in more rapid drain of the pulse generator energy supply, such as the stimulator battery. In the case of an IPG having a non-rechargeable battery, this may significantly shorten the useful life of the device. In the case of an IPG having a rechargeable battery, this may require more frequent recharging of the battery, which presents an inconvenience to the patient and which may also shorten the useful life of the device. Therefore, methods for reducing the number of stimulation channels are presented herein. While several stimulation channels may still be programmed, a reduced number of channels, such as one channel, may be used to deliver stimulation therapy to the patient.

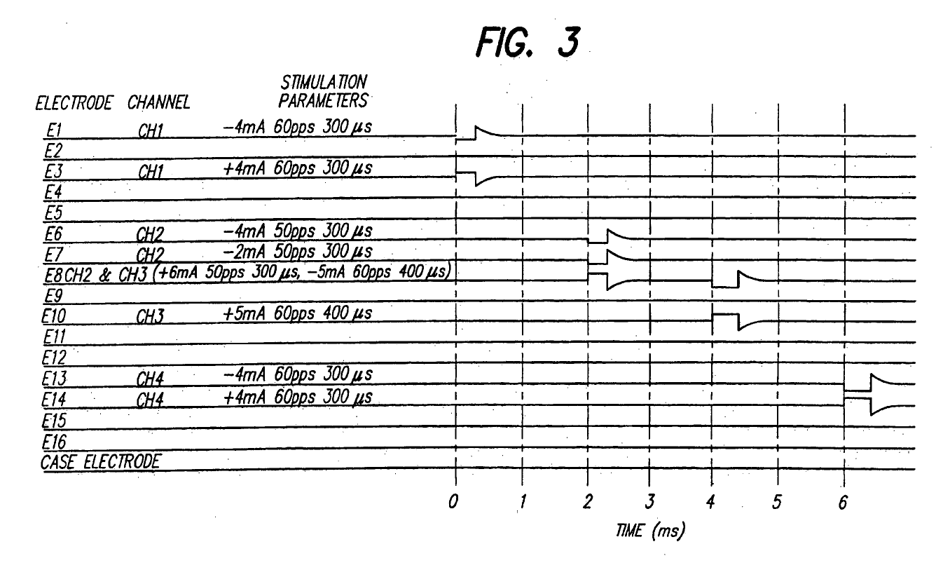

For example, the operation of multiple channels used to provide a stimulus pattern through multiple electrodes is illustrated in FIG. 3. FIG. 3 assumes the use of sixteen electrodes connected via one or more leads to an implantable pulse generator (IPG) capable of multiple-channel stimulation. In addition to these sixteen electrodes, which are numbered E1 through E16, a case electrode (or return electrode) is also available. In FIG. 3, the horizontal axis is time, divided into increments of 1 millisecond (ms), while the vertical axis represents the amplitude of a current pulse, if any, applied to one of the sixteen electrodes. Thus, for example, at time t=0 ms, FIG. 3 illustrates that a current pulse of 4 mA (milliamps) appears on channel 1 at electrode E1 and E3. FIG. 3 further shows that this current pulse is negative (−4 mA) on electrode E1 and positive (+4 mA) on electrode E3. Additionally, FIG. 3 shows that the stimulation parameters associated with this current pulse are set at a rate of 60 pulses per second (pps), and that the width of the pulse is about 300 microseconds (μs).

Still with reference to FIG. 3, it is seen that at time t=2 ms, channel 2 of the IPG is set to generate and apply a 6 mA pulse, having a repetition rate of 50 pps and a width of 300 μs, between electrode E8 (+6 mA) and electrodes E6 and E7 (−4 mA and −2 mA, respectively). That is, channel 2 of the IPG supplies a current pulse through electrode E8 (+6 mA) that is shared on its return path through electrode E6 (−4 mA) and electrode E7 (−2 mA).

As further seen in FIG. 3, at time t=4 ms, channel 3 of the IPG 100 is set to generate and supply a 5 mA pulse to electrode E10 (+5 mA) which is returned through electrode E8 (−5 mA). This pulse has a rate of 60 pps, and a width of 400 μs. Similarly, it is seen that at time t=6 ms, channel 4 of the IPG is set to generate and supply a 4 mA pulse to electrode E14 (+4 mA) which is returned through electrode E13 (−4 mA). This channel 4 pulse has a rate of 60 pps and a width of 300 μs.

The particular electrodes that are used with each of the four channels of the IPG 100 illustrated in FIG. 3 are only exemplary of many different combinations of electrode pairing and electrode sharing that could be used. That is, any channel of the IPG may be programmably connected to any grouping of the electrodes, including the reference (or case) electrode. When more than two electrodes are used with a given channel, the sum of the current sourced from the positive electrodes should be equal to the sum of the current sunk (returned) through the negative electrodes, as is the case with channel 2 in the example of FIG. 3 (+6 mA sourced from electrode E8, and a total of −6 mA sunk to electrodes E6 [−4 mA] and E7 [−2 mA]).

In the embodiment described above, it is thus seen that the SCS system may have sixteen electrodes, each of which is independently programmable relative to stimulus polarity and amplitude for each of up to four different programmable channel assignments (groups or phase generators). In operation, each channel identifies which electrodes among the sixteen electrodes, E1, E2, E3, . . . E16 and the IPG case electrode (reference electrode) are to output stimulation pulses in order to create an electric current field. All electrodes assigned to a given channel deliver their stimulation pulses simultaneously with the same pulse width and at the same pulse rate. In some embodiments, the IPG case electrode is programmable either as a Positive (i.e., a passive anode) or OFF for each channel. Thus, monopolar stimulation is provided when the only electrode programmed to Positive is the IPG case electrode, and at least one other electrode is programmed to Negative (i.e., a cathode). For each of the other electrodes, E1, E2, E3, . . . E16, on each channel, the polarity is programmable to Negative (cathode) with associated negative current amplitude, Positive (anode) with an associated positive current limit amplitude, or Off. In other embodiments, the case electrode may be programmed as a cathode.

In a preferred embodiment, the amplitude is programmable from −12.7 mA to +12.7 mA in 0.1 mA steps. The total simultaneous current capability from all of the anodes to all of the cathodes is at least 20 mA when operating at 130 Hz and with a 0.5 millisecond pulse width into an equivalent 500 ohms load. (Equivalent load means all cathodes ganged through a single 500 ohm load into all anodes ganged.) The programming of the total current capability into all cathodes while a given channel pulse is active is limited to the maximum IPG channel current capability.

As described, it is thus seen that any of the n electrodes may be assigned to up to k possible groups (where k is an integer corresponding to the number of channels.) Moreover, any of the n electrodes can operate, or be included in, any of the k channels.

An advantage of the present invention is the combining of the n electrodes into a minimum number of channels k. The combining may be accomplished during or after the programming steps. Thus, a combined single or reduced number of channels may ultimately deliver stimulation pulses to a patient during therapy. A reduced number of new stimulation combinations may induce two or more corresponding therapeutic effects on a patient's body. One example of a therapeutic effect is a paresthesia sensation in an area of the patient's body. Thus, delivery through a reduced number of channels may simultaneously induce paresthesia in two or more areas of a patient's body, which areas may or may not overlap.

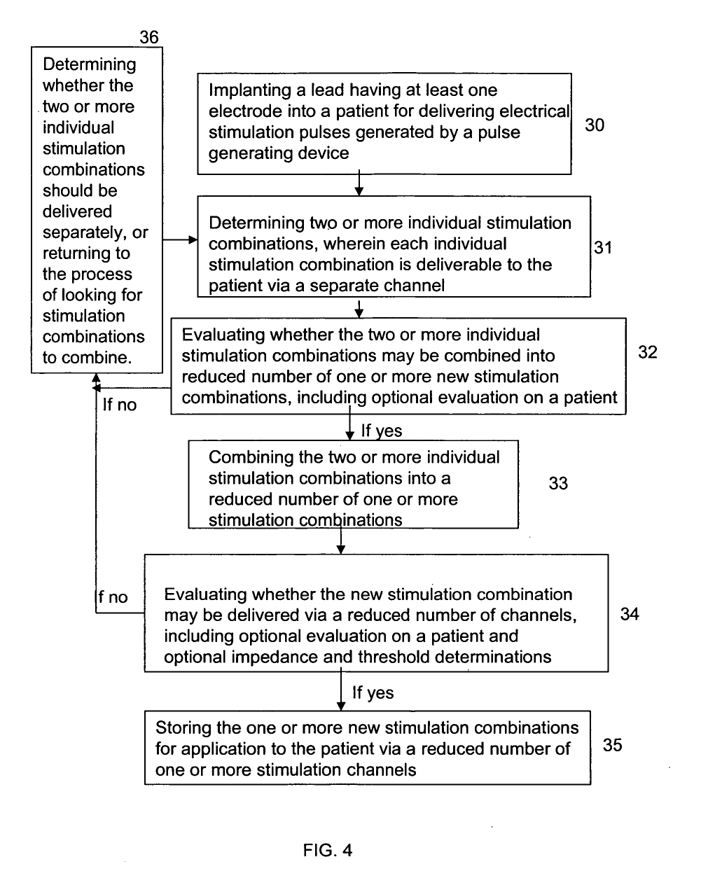

The group of electrodes in a channel may be referred to as a “stimulation combination.” FIG. 4 illustrates several steps that may be used to reduce a number of stimulation combinations into a minimum number of channels. In step 30, the hardware of a typical SCS system is described, wherein a patient may be implanted with a lead having at least one electrode for delivering electrical stimulation pulses generated by a tissue stimulator. As described, typical hardware also may include a programmer capable of communicating with the tissue stimulator.

Next, at step 31, two or more stimulation combinations are determined, wherein each stimulation combination corresponds to an effective therapy, such as the sensation of paresthesia in one or more areas of a patient's body. Each stimulation combination may have its own stimulation pulse amplitude, pulse width, and pulse duration, known as stimulation parameters. The stimulation combinations may be determined from an appropriate database that stores electrode configurations and possibly also associated paresthesia areas of the body, or may be determined as described in U.S. Pat. No. 6,052,624, earlier incorporated by reference. For an individual patient, two or more stimulation combinations may be determined based on testing various combinations on the patient in order to map the region of pain with the area of paresthesia. Such systems of creating and using a mapping database are described in U.S. Pat. Nos. 6,622,048; 6,393,325; 6,516,227, each herein incorporated by reference in its entirety. Thus, as an example, stimulation combination 1 may correspond to the upper leg, stimulation combination 2 may correspond to the lower leg and stimulation combination 3 may correspond to the lower back.

At step 32, the user may select the two or more stimulation combinations that will be evaluated for delivery to the patient at a given time. For example, the user may select stimulation combinations 1 and 3 to be delivered, in order to treat pain in both the upper leg and the lower back.

Also at step 32, the two or more stimulation combinations are evaluated to determine if they may be combined into a reduced number of stimulation combinations. Thus, the reduced number of stimulation combinations contains the selected collection of stimulation combinations that will be active for a period of time such that the therapy delivered by each stimulation combination is experienced simultaneously.

Several factors may be considered to determine if two or more stimulation combinations may be combined into a reduced number of stimulation combinations. If there is no overlap in the electrodes used by each stimulation combination and if the stimulation fields generated by each stimulation combination will have minimal interaction, then it is likely that the stimulation combinations may be combined into a reduced number of stimulation combinations. For example, Table 1 below illustrates two stimulation combinations in a patient having a single octapolar lead that can likely be combined into a reduced number of stimulation combinations, i.e., a single stimulation combination.

TABLE 1

| Stimulation combination | Area Name | Body Area covered |
|---|---|---|
| +, −, 0, +, 0, 0, 0, 0 | Area 1 | Upper Leg |
| 0, 0, 0, 0, 0, +, 0, − | Area 2 | Lower Leg |

In this example, the stimulation combination to supply therapy for the upper leg (Area 1) uses only electrodes 1, 2 and 4. The stimulation combination to supply therapy for the lower leg uses only electrodes 6 and 8. Thus, there is no overlap in the electrodes used by each stimulation combination.

At step 33, once the determination is made that two or more stimulation combinations may be effectively combined into a reduced number of channels, the two are combined. Taking the example of Table 1 above, the two stimulation combinations for Areas 1 and 2 are combined into a single combination (+, −, 0, +, 0, +, 0, −). The combining step may include a redistributing of current over the combined active electrodes, such as for instance, in a pro rata manner.

At step 34, a programmer evaluates whether the new stimulation combination may be delivered via a reduced number of channels. Because the stimulation field generated by the Area 1 stimulation combination will be centered nearer the electrode 1 end of the array and the stimulation field generated by the Area 2 stimulation combination will be concentrated nearer the electrode 8 end of the array, the two stimulation fields will have a minimal amount of interaction. Thus, these two stimulation combinations may likely be combined into a single channel of stimulation. Once the determination is made that a reduced number of channels may be used, the programmer stores the new stimulation combination to be delivered via the reduced number of channels, at step 35.

As is shown in FIG. 4 at step 36, if evaluations reveal that a new reduced stimulation combination is not effective or if delivery may not be accomplished through a reduced number of channels, the programmer returns to step 31 or determines to provide the stimulation without reducing the number of stimulation combinations or the number of channels. Determination may be made after step 32 and/or 34 to return to step 31 to continue to search for a reduced number of stimulation combinations or to supply the individual (uncombined) stimulation combinations to the patient.

Evaluation at steps 32 and 34 may include testing on a patient, programming logic, and other objective measurements. As one example, even if two stimulation combinations share one or more electrodes, they may, in come cases, be combined into a single channel using an algorithm that makes determinations as to the overall value of various electrode distributions. For example, suppose that two combinations, each having a pulse amplitude of 5 mA, share one particular electrode, where combination 1 uses the electrode for 4.5 mA or 90% of its cathodic current and combination 2 uses the electrode for 0.55 mA or 15% of its cathodic current. An algorithm may choose to use that electrode for combination 1 at an amplitude of 4.5 mA. This magnitude is sufficient to provide the cathodic current needed from this electrode for each of the stimulation combinations. The algorithm will then redistribute the 0.55 mA of anodic current from combination 2 either to an adjacent electrode or to an already-used anode in combination 2, in order to assure charge balance. Although such redistributions will affect the stimulation field, the algorithm can be programmed to find redistributions that will minimize the change to the stimulation fields. The algorithm may provide several different redistributions that may be tested to determine which produces the best acceptable result.

Several methods may be used by the algorithm to minimize the change in stimulation field. For example, it is known that in SCS, the location and amplitude of the cathodic current tends to have a greater and more focused effect on tissue depolarization and the resulting sensation of paresthesia. Thus, the algorithm should minimize cathodic changes and compensate using changes in anodic current distribution where needed. Previously unused electrodes may be programmed with cathodic or anodic current to mimic the desired combined stimulation field. Likewise, assessment of stimulation field interaction (including an assessment of the proximity of anodes and cathodes) once stimulation combinations are combined may include measuring impedance and therapeutic threshold values. The intent of this assessment is to determine if the energy savings that is obtained by combining electrode combinations is actually not achieved due to extraordinarily high impedance or therapeutic thresholds for the new combined combination. For example, in epidural spinal cord stimulation implantations, the electrode impedance will typically range between about 400 ohms and 1000 ohms. If a proposed program results in using electrodes which have very high impedances such that the energy drain from the battery is increased beyond that used by the uncombined combinations, delivering the stimulation combinations in a combined channel may not be desirable. Impedance measurement and its importance in stimulation systems are more thoroughly detailed in U.S. Pat. No. 6,516,227, earlier incorporated by reference in its entirety.

Likewise, perception and maximum threshold measurements may be important to evaluation at steps 32 and 34. As described in U.S. Pat. No. 6,393,325, several threshold measurements (e.g., four) may be taken, and the results may be interpolated to provide threshold levels for the entire implanted electrode array(s). These values may then be used as upper and lower bounds on redistributions implemented by the algorithm. For example, if a threshold measurement determines that the maximum comfortable stimulation level (threshold) for electrode 1 when used as a cathode is 4 mA, then this value should not be exceeded when redistributing current. Similarly, if a threshold measurement determines that the minimum stimulation level required for the patient to perceive paresthesia in the upper leg is 2 mA when electrode 7 is the cathode and electrode 6 is the anode, then redistribution of current should not result in stimulation levels dropping below these values.

Threshold measurements may indicate that certain stimulation combinations should not be delivered via a single channel. For example, if amplitude values for stimulating the right lower leg are in the order of about 15 mA and amplitude values for stimulation the right upper leg are in the order of about 2 mA, it may not be possible to deliver the electrode combinations via a single channel, even though there may be no overlap in the electrodes used to deliver the stimulation pulses. When the stimulation combinations require such radically different amplitude levels, incrementally increasing amplitude may have a very different effect in, e.g., the right lower leg and right upper leg. However, if threshold measurements are about the same order of magnitude, delivering the stimulation combinations via a single channel may be desirable.

Evaluations at step 32 and 34 may include a measurement of patient activity. For example, in SCS and other therapies, evoked potentials could be measured over the spinal cord or in the periphery or at the cortex. In motor disorders and other therapies, limb or extremity motion may be measured.

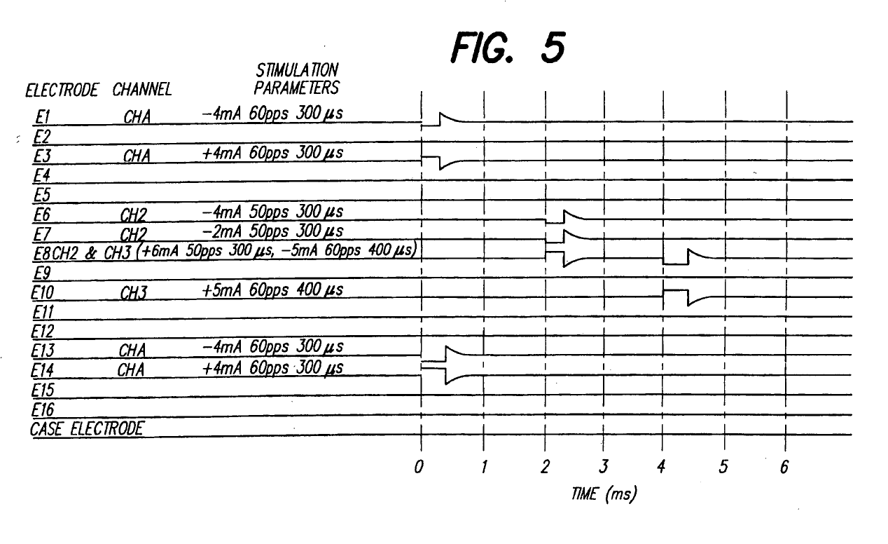

FIG. 5 is an illustration of the combination of stimulation combinations 1 and 4 from FIG. 3. In FIG. 3, stimulation combination 1 was provided on channel 1 using electrodes E1 and E3, while stimulation combination 4 was provided on channel 4 using electrodes E13 and E14. Because none of the electrodes are shared, and (assuming two parallel 8-electrode leads) because the stimulation fields are on different arrays (and at different ends of their respective arrays), it is unlikely that there will be significant interaction of the stimulation fields. Additionally, the stimulation parameters of the two stimulation combinations are identical. Thus, the two stimulation combinations can be combined into a single channel (new channel A).

In FIG. 5 the same stimulation is supplied as in FIG. 3, except that only 3 channels are used. New channel A uses electrodes E1, E3, E13 and E14. All four (4) of these electrodes output stimulation pulses at t=0 ms, as illustrated by the four (4) waveforms for E1, E3, E13 and E14. Thus, the original four (4) channels illustrated in FIG. 3 was reduced to the three (3) channels seen in FIG. 5.

Alternatively, even if it is determined by an algorithm that combining stimulation combinations is unlikely to produce an effective result, the stimulation combinations may still be combined into a single channel if effective for a patient. Thus, at step 34 of FIG. 4, the reduced number of channels may be tested on a patient. If the patient indicates that the single channel provides improved/effective stimulation to treat multiple areas, then that combination of stimulation combinations may be retained.

Several different redistributions may be tested to determine if one or more produce an effective result. For example, even if two stimulation combinations share one or more electrodes, they may still be combined into a single channel. As previously explained, two combinations may each have a pulse amplitude of 5 mA and share one particular electrode, where combination 1 uses the electrode for 4.5 mA or 90% of its cathodic current and combination 2 uses the electrode for 0.55 mA or 15% of its anodic current. The electrode as part of combination 1 and as part of combination 2 may be tested on a patient for determination if the electrode should act as a cathode in combination 1 or as a anode in combination 2 in the combined or reduced channel. As described, at step 32, these possible variations for shared electrodes may be tested on the patient.

The therapeutically effective stimulation combinations delivered through a reduced number of channels may be stored in a programmer for later use with the patient, at step 35. The resulting reduced stimulation combination may be programmed together to have a common stimulation pulse amplitude, pulse width, and pulse duration. Or, as noted at step 31, the stimulation parameters may be associated with the original stimulation combinations. Thus, in a stimulator that has unique current control of each electrode, the individual stimulation combinations may retain their original stimulation parameters within the new reduced stimulation combination. Various methods of selecting suitable values for these stimulation parameters are known and include the methods described, e.g., in U.S. Pat. No. 6,393,325, and U.S. Ser. No. 11/026,859, each herein incorporated by reference in its entirety. The selecting of suitable values for these parameters may be automated through use of suitable software or may be manually adjusted through a user interface.

The current amplitude may be selected to be at or near its maximum output, 20 mA, since this current may be distributed among a number of electrodes. Suitable values for pulse width and pulse duration may be selected and adjusted accordingly.

The methods of combining multiple stimulation combinations into a reduced number of channels may be used in connection with any type of stimulator. A stimulator with unique current control over each electrode may be used. The stimulation electric field generated by such stimulators is a superposition of the fields from individual combinations since the impedance of each electrode does not affect the delivered current.

The methods of the present invention may be incorporated into any tissue stimulation system, such as any SCS, neural, or muscle stimulation system. Thus, in another embodiment, a tissue stimulation system is provided. A system may comprise: (1) a pulse generating device for generating electrical stimulation pulses; (2) at least one implanted lead including at least one electrode for delivering the electrical stimulation pulses generated by the pulse generating device; and (3) a programmer for programming two or more stimulation combinations to be generated by the pulse generating device to be delivered via one or more stimulation channels. The programmer may be capable of combining the stimulation combinations into a reduced number of new stimulation combinations to be delivered by the at least one electrode over a reduced number of stimulation channels. The new stimulation combinations correspond to two or more corresponding therapeutic effects on the patient's body.

In SCS systems, the implanted lead is implanted near the patient's spinal column. As explained in the hardware description, the programmer may be incorporated into the pulse generating device or it may be incorporated into a separate device. The pulse generating device, such as an IPG, may implanted in the patient's body. The programmer may be implanted within or external to the patient.

The programmer may combine the stimulation combinations into a reduced number of new stimulation combinations during the programming of the individual stimulation combinations. Such combining may be most useful for systems having a single channel. Alternative to this simultaneous combining, the programmer may combine the stimulation combinations into a reduced number of new stimulation combinations after the programming of the individual stimulation combinations. For instance, programming may occur in multiple channels, but the new stimulation combinations are delivered over a reduced number of stimulation channels.

While the invention herein disclosed has been described by means of specific embodiments and applications thereof, numerous modifications and variations could be made thereto by those skilled in the art without departing from the scope of the invention set forth in the claims. For example, the methods discussed above are not limited to spinal cord stimulation systems and may be used with many kinds of stimulation systems such as, but not limited to, those described above, cochlear implants, cardiac stimulation systems, peripheral nerve stimulation systems, muscle tissue stimulation systems, brain stimulation systems and micro stimulators.

What is claimed is:

1. A method of reducing the number of stimulation channels used for a stimulation therapy, the method comprising:
determining two or more individual stimulation combinations, wherein each individual stimulation combination is capable of inducing a corresponding therapeutic effect in a patient's body and wherein each individual stimulation combination is deliverable to the patient via a channel;
evaluating the two or more individual stimulation combinations to determine if the two or more individual combinations may be combined into a reduced number of one or more new stimulation combinations;
combining the two or more individual stimulation combinations into a reduced number of one or more new stimulation combinations; and
storing the one or more new stimulation combinations for application to the patient via a reduced number of one or more stimulation channels.

2. The method of claim 1, wherein the evaluation includes evaluating overlap of active electrodes in the two or more individual stimulation combinations.

3. The method of claim 1, further comprising applying stimulation to the patient according to the one or more new stimulation combinations via the reduced number of one or more stimulation channels.

4. The method of claim 1, wherein the combining step includes redistributing current over active electrodes.

5. The method of claim 1, wherein if the two or more individual stimulation combinations share an active electrode, determining which stimulation combination uses the shared electrode for a greater amount of current distribution and assigning to the shared electrode that amount of current distribution.

6. The method of claim 1, wherein the evaluating includes measuring at least one of impedance and threshold values for the reduced number of one or more new stimulation combinations.

7. The method of claim 1, wherein the one or more new stimulation combinations correspond to two or more corresponding therapeutic effects on the patient's body.

8. The method of claim 7, wherein the reduced number of stimulation channels is a single channel.

9. A method of reducing the number of stimulation channels for a stimulation therapy, the method comprising:
determining two or more individual stimulation combinations, wherein each individual stimulation combination is capable of inducing a corresponding therapeutic effect in a patient's body and wherein each individual stimulation combination is deliverable to the patient via a channel;
evaluating the two or more individual stimulation combinations to determine if the two or more combinations may be combined into a reduced number of one or more new stimulation combinations;
combining the two or more individual stimulation combinations into the reduced number of one or more new stimulation combinations;
applying stimulation to the patient according to the new one or more stimulation combinations via a reduced number of one or more stimulation channels; and
evaluating the new one or more stimulation combinations.

10. The method of claim 9, wherein the evaluating the effectiveness of the new one or more stimulation combinations includes measuring at least one of impedance and threshold values.

11. The method of claim 9, wherein the new one or more stimulation combinations correspond to two or more corresponding therapeutic effects on the patient's body.

12. The method of claim 9, wherein the combining step includes redistributing current over active electrodes.

13. The method of claim 9, wherein if the two or more individual stimulation combinations share an active electrode, determining which stimulation combination uses the shared electrode for a greater amount of current distribution and assigning the shared electrode that amount of current distribution.

14. The method of claim 9, wherein the evaluating the effectiveness of the new one or more stimulation combinations includes evaluating patient feedback or performance.

15. A tissue stimulation system comprising:
a pulse generating device for generating electrical stimulation pulses;
at least one implanted lead including at least one electrode for delivering the electrical stimulation pulses generated by the pulse generating device; and
a programmer for programming two or more individual stimulation combinations to be generated by the pulse generating device to be delivered via one or more stimulation channels,
wherein said programmer is capable of combining the individual stimulation combinations into a reduced number of one or more new stimulation combinations to be delivered by the at least one electrode over a reduced number of one or more stimulation channels.

16. The system of claim 15, wherein the one or more new stimulation combinations correspond to two or more corresponding therapeutic effects on the patient's body.

17. The system of claim 15, wherein the implanted lead is implanted near the patient's spinal column.

18. The system of claim 15, wherein the programmer is incorporated into the pulse generating device.

19. The system of claim 15, wherein the programmer is external to the patient's body.

20. The system of claim 15, wherein the programmer is capable of combining the individual stimulation combinations into the reduced number of one or more new stimulation combinations during the programming of the individual stimulation combinations.

21. The system of claim 15, wherein the programmer is capable of combining the individual stimulation combinations into the reduced number of one or more new stimulation combinations after the programming of the individual stimulation combinations.
